(19) United States
(12) Patent Application Publication
(10) Pub. No.: US 2023/0253087 A1
(43) Pub. Date: Aug. 10, 2023

(54) System and Method for Self-Guided Physical Therapy

(71) Applicant: Michael Hashemi, Signal Hill, CA (US)

(72) Inventor: Michael Hashemi, Signal Hill, CA (US)

(21) Appl. No.: 17/666,520

(22) Filed: Feb. 7, 2022

(51) Int. Cl. G16H 20/30

(52) U.S. Cl. CPC G16H 20/30

(57) ABSTRACT

A system and method are provided for establishing self-guided physical therapy treatment enabled by a computing device. The digitally implemented system and method is performed by the computing device. The system displays and receives input from a user or patient regarding a variety of screening parameters comprising safety screening parameters, subjective screening parameters, and objective screening parameters. A user’s response input for objective screening parameters may comprise images or videos as prompted by the system. Responsive to and based on the user’s input for each of the screening parameters, the system and method generates a custom physical therapy treatment plan and presents physically therapeutic interventions for self-administration.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material that is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to the field of physical therapy, more particularly to a method and system for determining physical therapy intervention through a computing device.

### BACKGROUND

Physical therapy restores the body to its optimal condition by reducing pain, increasing mobility, and promoting healing. Unfortunately, physical therapy is unattainable for many because of time and cost constraints and therapist availability.

Medical treatment by physical therapy can be a substantial commitment for many who would benefit from it. Indeed, depending on the nature and severity of a particular physical ailment, physical therapy can require a patient’s attendance and participation in multiple sessions lasting between 30 minutes to an hour every per week. This time commitment does not even consider additional time constraints, such as a patient’s commute, time spent attempting to obtain an appointment with a practitioner, and time spent waiting to be seen by the practitioner, among others. Many prospective patients lack the time and do not get the needed treatment. Although the time commitment can be burdensome, extending the length of time between physical therapy appointments to reduce this burden may be associated with delayed feedback, progress, and healing.

One proposal to make medical treatment, in general, more attainable is telehealth. While telehealth allows a therapist and patient to engage one another while in different locations from one another and reduces travel and wait times, a shortage of qualified physical therapists makes getting even a telehealth appointment challenging on the basis of simple supply and demand. In addition, many telehealth proposals, such as U.S. Application Nos. 2020/0,066,414 to Neff and 2015/0278,453 to Joao and U.S. Pat. No. 9,974,485 to Robertson, still require live interaction between the health professional and patient. Thus, these proposals are deficient because they still require live interaction, which inherently limits the number of patients a health professional is able to treat. Therefore, access to physical therapy remains limited to many.

Physical therapy as a particular form of medical treatment may include a series of guided exercises to strengthen the body, and so some have proposed following certain exercise programs may be a sufficient alternative to physical therapy. One proposal for a remote exercise system is U.S. Pat. No. 8,221,292 to Barker, which teaches guided interaction with workout equipment. This proposal is deficient, though, because it requires the use of specialized equipment, and the guided interaction is limited to on-site, live, guidance. Thus, this proposal suffers from the same deficiencies as the proposal above. In contrast, U.S. Pat. No. 8,613,689 to Peloton Interactive Inc, teaches a self-guided exercise guidance system that is adaptable to fit an exercise program into the user’s schedule. However, it is still deficient because it does not provide custom training based on user needs.

Currently, no proposal teaches a system that provides a custom physical therapy program that reduces in-office interaction, which decreases the time burden on the healthcare system as well as individual therapists and the patients and makes physical therapy more attainable. Thus, there remains a need for an accessible system to provide custom physical therapy guidance.

### SUMMARY

The present disclosure is directed to a digitally implemented system and method for self-guided physical therapy. Broadly, the system is configured to receive user selections characterizing each of a plurality of screening parameters and assign physically therapeutic interventions for the user to perform based on parameter selections.

Physical therapy is the treatment of injury or disease by physical methods to regain or improve physical abilities. The present disclosure is a digitally implemented system and method which may allow a user to regain or improve physical abilities in a desired region, such as the location of an injury, or improve overall physical health.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In one embodiment, the system is operative to display a plurality of user-specific screening parameters comprising a combination of user-specific safety parameters, subjective parameters, and objective parameters.

In order to identify the user, the system is operative to receive biographical information including, but not limited to the user’s name, age, location, and/or contact information. A person of ordinary skill in the art will recognize other forms of biographical information exist and may be used in practicing the invention. In some embodiments, the biographical information may further comprise a referral such as instructions to seek or a script for physical therapy from a medical professional, internet form, or physical therapist. Thus, it is contemplated that the system may be operative to receive medical documents in addition to the user’s biographical information, such as the exemplary scripts or prescriptions for physical therapy.

In addition, the system may be operative to assign a unique user identifier to the biographical information. In such an embodiment, the unique user identifier may be the user’s name. In another embodiment, the unique user identifier may be a series of numbers, letters, or combinations thereof. The particular form taken by the unique user identifier will not limit the invention.

In some embodiments, the system is operative to authenticate that the user identity matches the referral.

Of the user-specific screenings, a safety screening may comprise safety parameters configured to determine a user’s program eligibility. That is, it is contemplated that certain physical or other health conditions may preclude users from participating in remote, self-guided physical therapy. In an exemplary embodiment, the safety parameters may characterize the user’s physical condition and comprise such information as the user’s injury history, temperature, or biological changes, such as changes in weight, continence, and mobility over a period of time, for example. A person of ordinary skill in the art will recognize many other safety screening parameters are available to practice the invention, and the foregoing are offered by way of example only and not limitation. As one example, it is contemplated that the user’s injury history could include information regarding past injuries in the same or different physical location as the current injury. As a clarifying example, it is understood that the user’s injury history may be used to determine other treatment areas, identify whether the injury is chronic, the expected level of recovery, and whether performance of certain self-guided interventions should be precluded. It is contemplated that the user’s temperature may indicate a fever or lack thereof which may, for example, provide an indication of infection. It is further contemplated that the user’s temperature may be classified at one of several levels, such as normal, warm, hot, and dangerous for example.

In some embodiments, the safety screening parameters may have a pre-assigned value. When a user selects any screening parameter, each associated pre-assigned value is assigned to the user, and if the combined value of the selected parameters is greater than a predetermined threshold, the system may be configured to alert the user that they are not eligible for self-guided physical therapy and further prevents the user from further interacting with methods for self-guided physical therapy enabled by the system. In such embodiments, the system may be further configured to transmit an alert to a medical professional. As an example, the safety screening may be configured to prevent further injury to the user and cause the system to display instructions to the user to seek crucial medical intervention. In another embodiment, when the pre-assigned values of each of the selected parameters are less than the predetermined threshold, the system may be configured to display a new set of user-specific screening parameters.

In an embodiment, a subjective exam may comprise subjective parameters operative to inform treatment or interventions directed at mitigating the user’s condition. In some embodiment, the subjective exam may also comprise subjective parameters operative to determine a user’s program eligibility. The subjective exam may comprise identification of pain location in reference to the user’s body and actions that give rise to increased or worsened symptoms, the time elapsed since the onset of pain, and pain level experienced by the user, for example. A person of ordinary skill in the art will recognize that these subjective exams are provided by way of example only and not limitation. Other subjective exams may be incorporated without departing from the invention. In some embodiments, the system is operative to assign a value to the subjective exam parameters and correlate the assigned value to predetermined values.

The user-specific screening parameters may further comprise parameters collected in an objective exam to determine treatment. In another embodiment, the objective exam may comprise subjective parameters operative to determine a user’s program eligibility. In such embodiments, the objective exam may include testing a user’s range of motion, flexibility, and strength in the affected area, for example. A person of ordinary skill in the art will recognize other forms of objective exams are possible as well. In some embodiments, the objective exam may be conducted by a medical professional. In other embodiments, the objective exam may be conducted by the user themself. It is contemplated that in a user-conducted objective exam, the system is operative to extract the parameters from a series of images or videos taken by the user or by some third party. In some embodiments, the objective parameters may be received in a plurality of forms. In some embodiments, the system is operative to assign a value to the objective exam correlating to predetermined values. As a clarifying example, the predetermined values may correlate to a user’s degree of motion in a certain body part, the force exerted by the user, and any angle of joint flexibility. In some embodiments, a higher value may be associated with each of a higher degree of motion, a higher force exerted, and a higher range of joint flexibility. A value of zero may be associated with an inability to move, exert force, or flex a joint, for instance. The same may be said for the subjective parameters as well.

In some embodiments, the values of the subjective exam may affect the values of the objective exam.

In one embodiment, the system is operative to correlate at least one intervention to the selected screening parameters. Further, the system is operative to assign at least one correlated intervention to the user for self-guided performance. In another embodiment, the system may be operative to transmit any assigned interventions and further receive approval of any assigned interventions to and from, respectively, a second electronic device associated with a physical therapist or another qualified medical professional. In some embodiments, the system may be operative to assign one or more interventions received directly from a second electronic device associated with a physical therapist or another qualified medical professional. One of ordinary skill in the art will recognize that the system, and in particular, any electronic device comprising the system, is further operative to display any assigned interventions.

In one embodiment, the system is operative to assign the user a physical therapist for remote oversight of self-guided performance of any assigned interventions.

In another embodiment, the system is operative to receive the user’s goals. For instance, as non-limiting examples, the user’s objective goals may include weight loss, increased muscle mass or strength, and higher energy levels, increased walking duration and/or speed, and/or the like. In such an embodiment, interventions may be assigned based on the user’s goals.

In one embodiment, the system is operative to collect the received user-specific parameters and identify and/or confirm an injury as reason to utilize the system for self-guided physical therapy. In a further embodiment, the user-specific parameters and identified and/or confirmed injury may be compiled in a report. In such an embodiment, the report may be sent to a third party. For example, the third party may be a physical therapist, medical services provider, employer, lawyer or law office, judicial body, or insurance company.

In one embodiment, the system is operative to receive progress updates. In such embodiments, interventions may be assigned based on the progress updates. In another embodiment, an updated report may be sent to the third party.

In another embodiment, the system is operative to record user interaction with the system.

In a further embodiment, the system may comprise a graphical user interface that displays an intervention. In such an embodiment, the graphical user interface may be configured for inputting user information in the account portion. In another embodiment, the graphical user interface may be configured for inputting medical information. For example, medical information may include symptoms, referral, and pain location. In a further embodiment, the method may include a step to receive further information. In another embodiment, the method may further include displaying new interventions.

Several advantages of the system and method are that they:
a.) increase accessibility to physical therapy;
b.) reduce the time commitment for physical therapy;
c.) reduce the burden on the healthcare system by resolving physical issues before requiring attention of additional medical professionals;
d.) provide more frequent access to physical therapy; allow a patient access to physical therapy in their home or other self-selected location;
e.) customize treatment plans to promote healing and other user-specified goals;
f.) reduce or, in some cases, eliminate the need for onsite or real-time supervision; and
g.) provide progress reports to a medical professional for remote oversight.

Thus, it is an object of this system and method to provide access to physical therapy through a custom, electronically implemented, and self-guided physical therapy program.

It is another object of this system and method that the user may access the physical therapy program without the need for direct physical therapist supervision.

It is yet another object of this system and method that the user may access the physical therapy program in the comfort of their home or any other location as desired.

It is an object of this system and method to reduce the time burden of receiving physical therapy on patients. It is a further object, to reduce the client burden on physical therapists.

It is an object of this system and method to provide a custom program to each user’s needs and goals.

It is a further object that the user may use the program to promote overall health.

It is another object of this system and method to be used in accordance with medical professionals.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 is a block diagram of a networked environment in which an exemplary embodiment of a system for self-guided physical therapy is implemented;

FIG. 2 illustrates an exemplary embodiment of a computing device shown in FIG. 1;

FIG. 3 is a flowchart depicting an embodiment of a method for determining physical therapy intervention;

FIG. 4 illustrates an alternative networked environment in which another embodiment of a system for self-guided physical therapy is implemented;

FIG. 5 illustrates an alternative networked environment in which another embodiment of a system for self-guided physical therapy is implemented;

FIG. 6 networked environment in which another embodiment of a system for self-guided physical therapy may be performed on a server;

FIG. 7 illustrates an embodiment of a method for self-guided physical therapy utilizing a referral;

FIG. 8 illustrates an embodiment of a method for self-guided physical therapy utilizing user-specific goals;

FIG. 9 illustrates an embodiment of a method for self-guided physical therapy and providing a report to a physical therapist; and

FIGS. 10A-B illustrates an embodiment of a method for self-guided physical therapy from a referral, confirming an injury, and providing a report to a third party.

One embodiment of the invention is implemented as a program product for use with a computer system. The program(s) of the program product defines functions of the embodiments (including the methods described herein) and can be contained on a variety of computer-readable storage media. Illustrative computer-readable storage media include, but are not limited to: (i) non-writable storage media (e.g., read-only memory devices within a computer such as CD-ROM disks readable by a CD-ROM drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present invention, are embodiments of the present invention. Other media include communications media through which information is conveyed to a computer, such as through a computer or telephone network, including wireless communications networks. The latter embodiment specifically includes transmitting information to/from the Internet and other networks. Such communications media, when carrying computer-readable instructions that direct the functions of the present invention, are embodiments of the present invention. Broadly, computer-readable storage media and communications media may be referred to herein as computer-readable media.

In general, the routines executed to implement the embodiments of the invention, may be part of an operating system or a specific application, component, program, module, object, or sequence of instructions. The computer program of the present invention typically is comprised of a multitude of instructions that will be translated by the native computer into a machine-readable format and hence executable instructions. Also, programs are comprised of variables and data structures that either reside locally to the program or are found in memory or on storage devices. In addition, various programs described hereinafter may be identified based upon the application for which they are implemented in a specific embodiment of the invention. However, it should be appreciated that any particular program nomenclature that follows is used merely for convenience, and thus the invention should not be limited to use solely in any specific application identified and/or implied by such nomenclature.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

The terms “person,” “party,” and the like should be broadly understood to refer to any person, entity, organization, or group.

The term “intervention” should be understood as actions and tasks relating to physical therapy. “Intervention,” “task,” and “treatment” may be used interchangeably.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications, and equivalents included within the spirit and scope of the disclosure as defined by the appended claims.

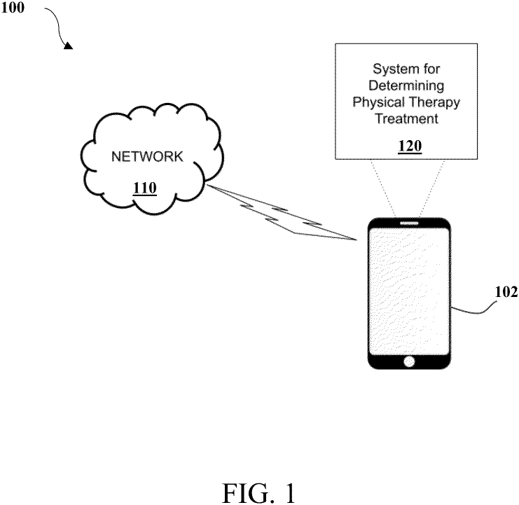

A description of an embodiment of a method and system for determining physical therapy intervention is now described followed by a discussion of the operation of various components within the system. In this regard, FIG. 1 illustrates an exemplary embodiment of the system for self-guided physical therapy 100 which includes at least one computing device. By way of example only and not limitation, FIG. 1 illustrates the system on one computing device 102, which may be associated with a user of the system as a physical therapy patient.

The computing device 102 includes a physical therapy system 100, which can be implemented in numerous ways such as, for example, and without limitation, an application executed on the computing device. The computing device 102 may be embodied, for example and without limitation, a tablet, desktop computer, or smartphone. The network can use one or more of various communications types such as, for example and without limitation, Wi-Fi, cellular, Bluetooth, dial-up, or Ethernet connection.

Users of computing device 102 may use their computing device to interact with the system, which enables them to access custom physical therapy treatment based on user-provided information.

In this exemplary embodiment, the system for self-guided physical therapy is hosted on the computing device 102.

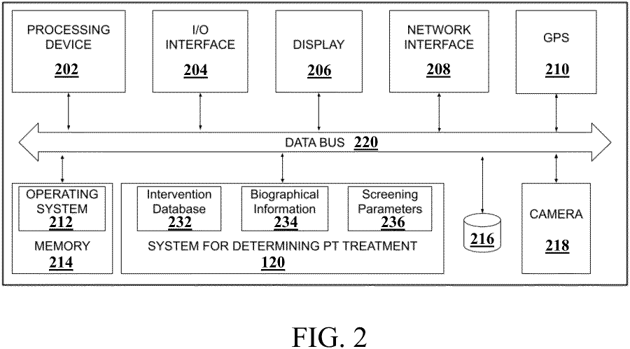

FIG. 2 illustrates computing device 102 shown in FIG. 1. As described earlier, computing device 102 may be a tablet, desktop computer, or smartphone but may also be embodied in any one of a wide variety of wired and/or wireless computing devices. As shown in FIG. 2, mobile device 102 includes a processing device (processor) 202, input/output interfaces 204, a display 206, a network interface 208, a GPS 210, a memory 212, and operating system 214, a mass storage 216, and a camera 218, with each communicating across a local data bus 220. Additionally, computing device 102 incorporates a system for physical therapy 100, which is depicted as including intervention database 232, biographical information 234, and screening parameters 236, although the location of information 232, 234, and 236 could vary.

The processing device 202 may include any custom made or commercially available processor, a central processing unit (CPU) or an auxiliary processor among several processors associated with the mobile device 102, a semiconductor based microprocessor (in the form of a microchip), a macroprocessor, one or more application specific integrated circuits (ASICs), a plurality of suitably configured digital logic gates, and other electrical configurations comprising discrete elements both individually and in various combinations to coordinate the overall operation of the system.

The memory 214 can include any one of a combination of volatile memory elements (e.g., random-access memory (RAM, such as DRAM, and SRAM, etc.)) and nonvolatile memory elements. The memory typically comprises native operating system 212, one or more native applications, emulation systems, or emulated applications for any of a variety of operating systems and/or emulated hardware platforms, emulated operating systems, etc. For example, the applications may include application-specific software which may comprise some or all the components of the computing device 102. In accordance with such embodiments, the components are stored in memory and executed by the processing device. Note that although depicted separately in FIG. 2, the system and method for self-guided physical therapy treatment 100 may be resident in memory such as memory 214.

For some embodiments, the computing device 102 will comprise GPS 210 or other means to determine the location of the computing device 102. One of ordinary skill in the art will appreciate that some physical therapists and medical professionals may be certified, permitted, or otherwise qualified to treat patients in limited geographic regions. Thus, providing a patient location via GPS 210 may inform whether a particular physical therapist or medical professional may be assigned in this respect to review any reports or provide remote oversite of the self-guided physical therapy as enabled by the system. Biographical information 234 including a patient user’s geographic location may serve this purpose as well.

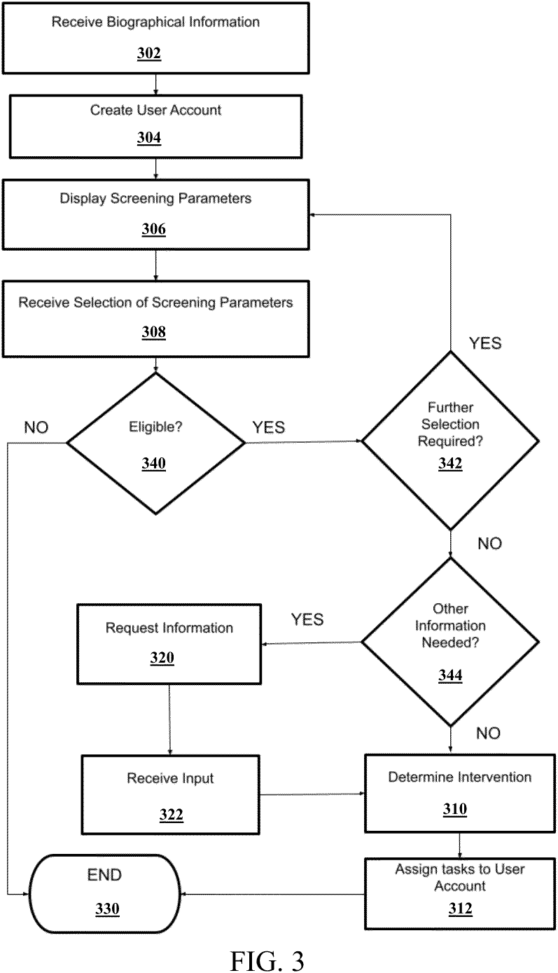

One of ordinary skill in the art will appreciate that the memory 314 can, and typically will, comprise other components which have been omitted for purposes of brevity. Note that in the context of this disclosure, a non-transitory computer-readable medium stores one or more programs for use by or in connection with an instruction execution system, apparatus, or device. With further reference to FIG. 3, network interface device 208 comprises various components used to transmit and/or receive data over a networked environment such as depicted in FIG. 1. When such components are embodied as an application, the one or more components may be stored on a non-transitory computer-readable medium and executed by the processing device.

FIG. 3 is a flowchart depicting an exemplary embodiment of a system and method for self-guided physical therapy treatment. As shown in FIG. 3, the method includes the steps of: receiving biographical information 302, creating a user account 304, displaying screening parameters 306, receiving a selection of screening parameters 308, determining eligibility 340, determining the necessary intervention 310, and assigning interventions to the user account.

In an embodiment, the biographical information 302 may comprise information relating to the user’s name, age, height, weight, geographical location, or contact information, however, a person of ordinary skill will recognize other forms of biographical information. The biographical information 302 may further comprise a referral to perform physical therapy from a medical professional, such as a doctor or physical therapist, internet form, court, employer, or insurance company.

The system may be operative to receive the biographical information 302 through user input or by extracting the data from another source such as the user’s medical records or the referral. As noted above, the user’s geographical location may be determined using GPS 210.

In an exemplary embodiment, creating the user account 304 may be accomplished by assigning the biographical information to a unique identifier. For example, the user’s name may be assigned as the unique identifier. Additionally, the unique identifier may be a series of numbers, letters, or a combination of numbers and letters. The unique identifier may identify the specific user on a variety of computing devices 102.

The displayed screening parameters 306 may include questions based on the user’s symptoms. In one embodiment, the screening parameters may be designed as a safety screening. The safety screening may include questions relating to the user’s previous injuries, biological changes, such as weight gain or loss, continency, or the user’s present physical conditions, including temperature.

In another exemplary embodiment, the displayed screening parameters 306 may be an objective exam. The objective exam may be characterized by quantitative data. For example, only and without limitation, the objective exam may comprise questions relating to the user’s actual, measurable a range of motion, flexibility, strength, and other forms of quantitative data. In some embodiments, the system may operative display reference photos demonstrating possible answers or results of the objective exam. For instance, a series of photos illustrating objective levels of flexibility may be displayed for selection by the user. As another example, the system may be configured to prompt a user to perform certain motions to objectively ascertain these measures. As one non-limiting, clarifying example, the user may be prompted to perform, a supine hip or knee extension and/or a supine hip or knee flexion to objectively determine the extent to which a user is capable of extending or flexing a particular body part. As another non-limiting, clarifying example, the system may prompt the user to sit or stand and perform a forward fold from their hips to objectively gauge hamstring length and flexibility. Other non-limiting exercise performances may be incorporated into the objective exam as well, such as those directed to ascertaining a user’s functional strength. Such exercises, known to those of ordinary skill in the art, may comprise, for instance, speed, agility, and quickness drills; unilateral and/or bilateral bridges; straight leg raises; bilateral and unilateral sit to stand tests; and even varieties of squat exercises.

As one non-limiting example of an objective exam offered for the sake of clarifying one aspect of the invention, in an embodiment where a user reports low back pain, the objective exam may comprise selections characterizing the user’s sitting posture such as whether the user’s sitting posture presents an objectively neutral or flexed spine when feet are flat on the ground, crossed at the knees or otherwise; or whether the user’s gait presents any noticeable limp, lean, or deviations. The objective exam in case of low back pain may further comprise means for testing neural mobility such as repeated ipsilateral side bends or extensions, a piriformis test, a lump test, and a lower nerve tension test, all known to those of ordinary skill in the art. Likewise, the user’s low back range of motion, flexibility, and mobility may be subject to the objective exam as well. Such objective exam may comprise selections directed to the user’s spine flexion and extension, hip flexion, and hamstring flexion in a sit and reach exercise. Results of any user selections in the exemplary objective exam may, of course, be correlated with certain interventions, discussed in more detail below.

The objective exam may be conducted by the user themself or by a medical professional. In instances when the objective exam is conducted by the user, the system may guide the user on how to conduct the exam. In such an embodiment, the user may input the data themselves. By way of example and not limitation, the system may instruct the user to lift an arm. In instances when the user is inputting the data themselves, the user may be instructed to measure the distance from the floor to the arm. The system will then receive 308 the user’s measurements.

In another such embodiment, the user may upload a series of images or videos of themselves performing movements or exercises, as prompted by the system in the objective exam, and the system will extract the necessary data. For example and not limitation, the system may receive 308 a video of the user lifting their arms. The system may then take that video and extract data relating to the user’s range of motion.

In instances when the objective exam is conducted by a medical professional, the data may be extracted from the referral. In another instance, the medical professional may enter the data into computing device 102 or another computing device communicatively linked to computing device 102 within the system.

In another embodiment, the displayed screening parameters 306 may be a subjective exam. The subjective exam may comprise information relating to the user’s current pain level and symptoms. For example, and not limitation, the subjective exam may include information relating to the pain location; pain levels; time since pain onset; and any aggravating factors that give rise to increased pain, functional limitations, such as whether the pain prevents the user from walking on flat, inclined surfaces, or stairs; squatting; running; participating in sports; and even performing employment duties. .In some embodiments, the subjective exam may further comprise specific concerns characterizing the pain or injury such as buckling, catching, locking, and even aggravation of prior injuries. Examples of selections that the system will receive 308 for subjective exams may include numbers, pre-listed actions, or user-inputted descriptions. In some embodiments, the system may be configured to display, by the computing device 102, a chart or image of a human body showing potential locations which may be further configured to receive selection by the user to indicate their own pain location. The system may then further prompt the user to input or select associated pain ratings, and any other information to the subject exam according to the foregoing descriptions.

In some instances, the screening parameters 306 may comprise a combination of safety screening, subjective exam, and objective exam parameters.

In one embodiment, eligibility 340 is determined by values assigned to the received parameter selections 308. Different values may be accorded to each parameter within each exam type. In some embodiments, each value may correspond to the user’s impairment as to each exam parameter. In some embodiments, the values may be provided on a numeric scale. For instance, and with respect to the subjective exam in particular, a value of 0 may be associated with no impairment - or no pain, full range of motion, and the like. A value of 1 may be associated with mild impairment, while a value of 2 may be associated with moderate impairment, and a value of 3 may be associated with notable impairment. Likewise, the time since symptom onset, symptom location, and any specific concerns may be assigned their own value. In some embodiments, the subjective exam will inform the type of injury, pain or impairment experienced by the user, and such type of injury, pain, or impairment may itself be assigned a value. As an example, the user input prompted in the subjective exam may be correlated with the following impairments: coordination or power deficiency, mobility deficiency, radiating pain, and generalized radiating pain, each associated with their own values.

Likewise, safety screening parameters may be associated with values as well. In some embodiments, higher values may be associated with greater risk to the patient and potential ineligibility to participate in self-guided physical therapy implemented by the system. For example, the values may be assigned to each parameter on a ten-point scale. If the system receives user input that the user may be particularly physically vulnerable or otherwise at risk of further injury absent real-time, in person, and professional oversight, then the system may assign a higher value to such parameters. Parameter responses associated with such higher risks or vulnerability may comprise, for example only and not limitation, a high temperature which may be associated with illness, high impact injury, and pain levels causing complete immobility. These and other parameter responses may be associated with a value of 10, corresponding to the highest risk of further injury or vulnerability. In contrast, parameter responses such as, for example only and not limitation, unexplained weight gain or a history of cancer, which may or may not result in irreparable injury to the patient over the course of self-guided physical therapy, may have an assigned value of five or lower.

If the assigned value of the parameters is greater than or equal to a pre-determined threshold the system may be operative to prevent further access to the user. As a further example, if a high fever has a value of ten, this section would prevent further access. That is, rather than assigning treatment, further interaction with the user’s account is prevented. It is contemplated that at certain values, the system may be operative to prompt further investigation or official diagnosis from a physician. Thus, in accordance with one exemplary embodiment, the system may be operative to display a message may be at computing device 102 which advises the user to seek assistance from a physician or other medical professional. In instances where a referral was provided, a notice may be sent to the source of the referral informing the source that the user is ineligibile for self-guided physical therapy under the system.

In one embodiment, all screening parameters are displayed 306 and received 308 before eligibility 340 is determined.

In one embodiment, safety parameters may be displayed and selections received to determine eligibility before any objective or subjective screening parameters are displayed or received. It is contemplated that this will prevent undue injury to users of the system. That is, eligibility 340 may be determined after a selection of less than all screening parameters 308. In such an embodiment, if the value of the selection of less than all the parameters is below the pre-determined threshold then more screening parameters may be displayed 306. The screening parameters may be displayed until all selections have been made or the value of the selections is greater or equal to the predetermined threshold.

If all screening parameters have been selected and the value of the selections is less than the predetermined threshold, additional information may be requested 320. For example, and not limitation, the other information 344 may be the user’s goals, payment information, questionnaires, or notice of equipment available for use by the patient in self-guided performance of any interventions assigned by the system. In some instances, no additional information may be needed, and an intervention 310 correlated to the user’s parameter selections will be assigned by the system to the user.

Intervention may be determined 310 based on the received information 308 and 322. The determined intervention 310 may then be assigned to the user account 312.

In some embodiments, system 300 may comprise a graphical user interface. In some instances, the graphical user interface is operative to receive input 302, 308, and 322. In other instances, the graphical user interface is operative to display the assigned interventions 310 as well as any parameters and prompts noted above.

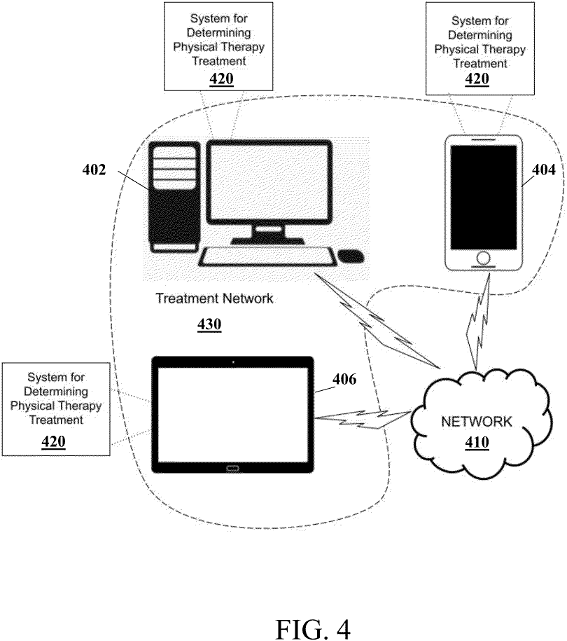

FIG. 4 is illustrative of an alternative networked environment in which another exemplary embodiment of a system for self-guided physical therapy treatment 400 is implemented. FIG. 4 illustrates three computing devices 402, 404, and 406 communicatively coupled via a network 410. Each of these computing devices may be, for example, a smartphone, tablet, laptop computer, or desktop computer. The network can use, for example and without limitation, Wi-Fi, cellular, Bluetooth, dial-up, or Ethernet connection.

Users of the computing devices 402, 404, and 406 may be, for example and without limitations, one or more of any of a patient, medical professional, insurance company, or court.

The treatment network 430 enables members to use their devices 402, 404, and 406 to interact with the system 400. In one embodiment, the treatment network 430 is created by a first computing device 402. In such embodiment, a second computing device 404 may join the treatment network 430. In some instances, the second computing device may join the treatment network 430 by providing the unique identifier associated with the user account on the first computing device. 402. In some such embodiments, the user associated with the unique identifier may be able to confirm or deny access. Upon receiving access, the second computing device 404 may be given access to information associated with the user’s account. For example and not limitation, the accessing user may receive access to the assigned interventions.

In instances when a referral is provided, the treatment network 430 may include the referral source.

In one embodiment, the users may consist of a patient and medical professional, such as a physical therapist. In such instances, system 400 may allow for a physical therapist to provide physical therapy without a patient coming into the office. For example, at a second computing device, a physical therapist may review and suggest further interventions on a user’s account during business hours and the patient may access at their convenience. In addition, the physical therapist may review patient information in a short amount of time, freeing up the physical therapist to engage more patients.

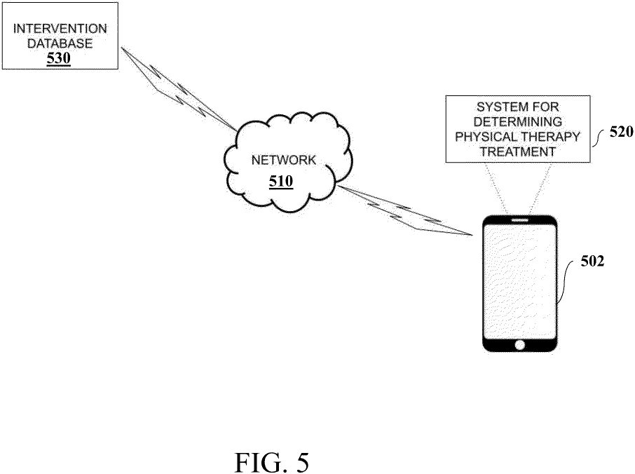

FIG. 5 illustrates an exemplary embodiment of the system for self-guided physical therapy treatment. As shown in FIG. 5, the system comprises a computing device 502 coupled to an intervention database 530. The computing device 502 may be, without limitation, a smartphone, tablet, laptop computer, or desktop computer. The network can use one or more various types such as, without limitation, cellular, Wi-Fi, Bluetooth, and Ethernet.

The intervention database 530 can comprise or store interventions assigned to the user account. Examples of interventions include, but are not limited to, strength training, cardiovascular and cardiopulmonary endurance, exercises to improve range of motion and/or flexibility, balance, breathing, and education. Educational intervention may address posture, load reduction techniques and strategies, and left/right motor discrimination. In some embodiments, interventions may be progressive or altered based on the patient’s changing needs. For instance, if an intervention comprises range of motion and/or flexibility exercises, the system may assign an intervention at a level associated with the subjective and objective exam. One particular example may involve knee flexibility. In such embodiments, a low level of initial knee flexibility or range of motion may correspond to a to a low intensity, easy, or “level 0” exercise. For instance, an intervention assigned in response to parameters indicating low knee range of motion or flexibility may be sitting knee flexion, known to those of ordinary skill in the art. As range of motion and flexibility improves, as determined by the various screening parameters, the system may assign progressively challenging interventions. In the example of knee flexibility or range of motion, a moderate or “level 1” exercise may be a heel slide with a strap, and a challenging or “level 2” exercise may be a prone knee bend. Thus, it will be recognized that whether progressively challenging interventions are assigned may be the result of selections made in repeated objective and subjective screenings.

Returning to the exemplary low back pain introduced above, interventions may comprise various stretches correlated to parameter selections received by the system as a result of the objective exam. For instance, interventions correlated to correcting power deficiencies and increasing strength to support posture and reduce back pain may comprise core strengthening limb flexion and extension exercises, and bird dog and/or plank exercises held for a period of time; spine extensor exercises such as prone press ups, cervicothoracic extensions, and prone superman exercises maintained for a period of time; and hip and/or knee strengthening exercises such as bi-and unilateral bridges and bi- and unilateral sit to stand exercises. Any or all of these interventions may be performed progressively according to repeated objective exams.

As another example, cardiovascular or cardiopulmonary interventions may comprise endurance-based exercises such as walking, jogging, running, rowing, and biking. These may be performed indoors, outdoors, in a gym, and with equipment as desired. The specific mode of a user’s performance of any assigned intervention will not limit the invention.

As another example, strength related interventions may progress from any speed, agility, and quickness exercises known to those of ordinary skill in the art at a first level, straight leg raises at a second level, sitting to standing exercises at a third level, various forms of lunges at a fourth level, and “monster walks” known to those of ordinary skill in the art at a fifth level.

As yet another example, balance or proprioceptive interventions may comprise sensory challenges and prompts to perform certain movements with a staggered stance.

As still another example, interventions may be directed to improving posture and movement and may comprise the display of ergonomic education such as videos, literature, and photos. Such interventions may comprise, where indicated by parameter selections, load reduction strategies, instructions to apply ice, heat, medication, self-stimulation, pacing, and graded progression. Of course, these may be remotely overseen by a physician or other qualified party at a second computing device.

Indeed, it is contemplated that any or all interventions may comprise displaying, at a computing device associated with the user, additional educational material or referral for the user to review or seek further interventions including but not limited to in-person care from a physician or specialist, or in-person interventions such as acupuncture, or even the procurement and use of an assistive device such as foot orthotics.

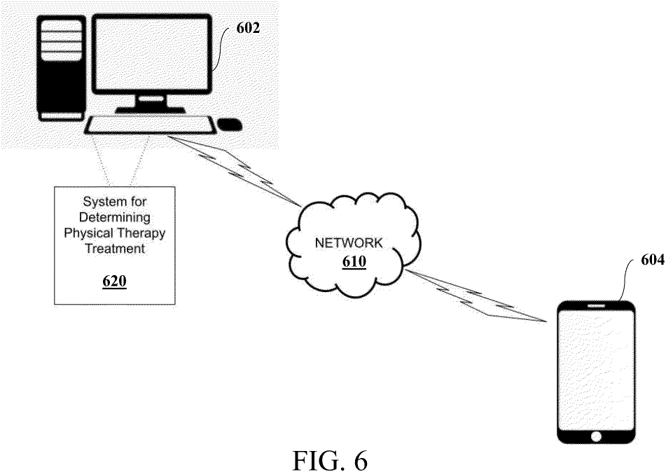

FIG. 6 is illustrative of an alternative networked environment in which another exemplary embodiment of a system for self-guided physical therapy. As shown in FIG. 6, the system comprises computing device 602. The computing device 602 may be, without limitation, a smartphone, tablet, laptop computer, or desktop computer. The network can use one or more various types such as, without limitation, cellular, Wi-Fi, Bluetooth, and Ethernet.

In this exemplary embodiment, a server 630 is operative to implement the system for physical therapy treatment 620 Specifically, server 630 is operative to implement the steps outlined in FIG. 3. Accordingly, the computing device 602 can access server 630 using and obtain information regarding physical therapy treatment.

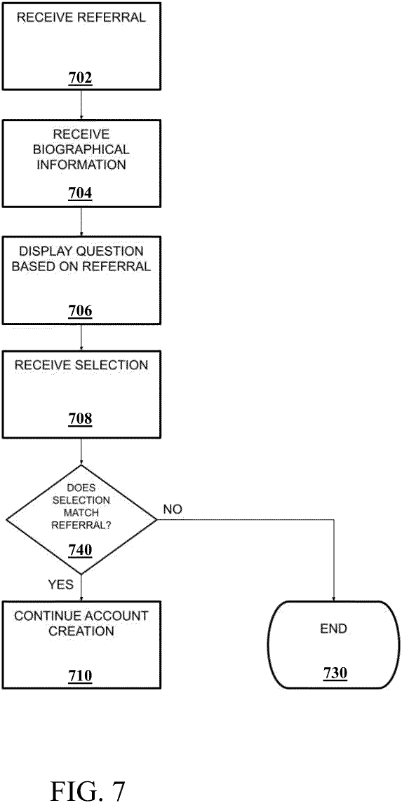

FIG. 7 is a flowchart depicting an exemplary embodiment of how a method for self-guided physical therapy utilizing a referral. As shown in FIG. 7, the method includes the steps of: receiving a referral 702; receiving biographical information 704; displaying questions based on the referral 706; receiving user’s selection 708; and verifying that the selection matches the referral 740. If the selection matches the referral, a user account may be created 710. If the selection does not match the referral, then the system may disregard the referral 730. In some embodiments, the system may be operative to generate a user account without any referral. In another embodiment, access to the system will be prevented.

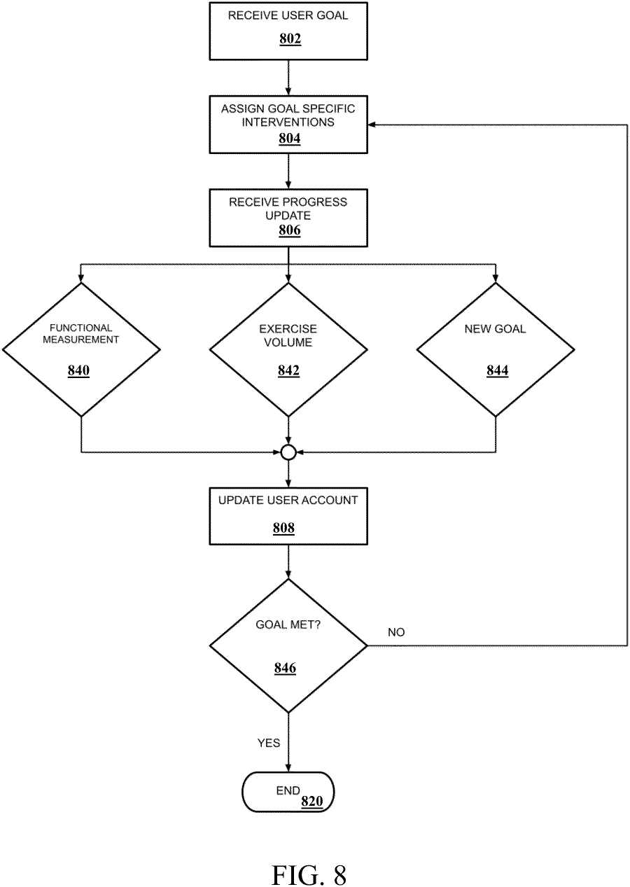

FIG. 8 is a flowchart depicting an exemplary embodiment of how a method for physical therapy treatment may utilize user-specific goals. As shown in FIG. 8, the method includes the steps of: receiving the user’s goal 802; assigning goal-specific interventions 804; receiving progress updates from the user 806; and updating the user account 808. Progress updates may include functional measurements, such as a functional reach test, timed up and go test, Berg balance scale, or six-minute walk test, all known to those of ordinary skill in the art, for example. Additionally, progress updates may include information relating to exercise volume, for example and not limitation, including reps performed, time spent working out, and exercises per week. In some embodiments, selections made in response to the subjective and/or objective exam may define baseline measurements for progress tracking. For instance, an increase or decrease in user-selected aggravating factors, comparison of functional limitations, and a change in pain values over time may inform a system user’s progress under self-guided physical therapy. In some embodiments the system may prompt a user to input selections and therefore receive selections in a questionnaire such as a fear avoidance belief questionnaire known to those of ordinary skill in the art.

In some embodiments, the user goals of FIG. 8 may include physical therapy goals, for example and not for limitation, including strengthening, weight loss, flexibility, and cardiovascular fitness.

In one embodiment, the progress updates 806 may be received periodically. For example, and without limitation, periodically may mean every time the user accesses the system, every week, every two weeks, every month, or other periods of time as necessary.

In some embodiments, the user may input a new goal 844 during the course of an intervention. In one instance, the new goal may be related to the original user goal, for example, the received goal 802 may have been to lose five pounds and the new goal 844 may be to lose ten pounds. In another instance, the new goal may be unrelated to the original user goal. For example, and not for limitation, the received goal 802 may have been to lose five pounds and the new goal 844 may be for the user to increase their knee’s range of motion to 140°.

If the user has met their goal 846 and has not inputted a new goal 844 then the system may end 820 and not assign further interventions. If the user has not met all their goals, then the system may assign further interventions 804. The system is operative to continue to assign further interventions until the user has met all user-specified goals 802 and 844.

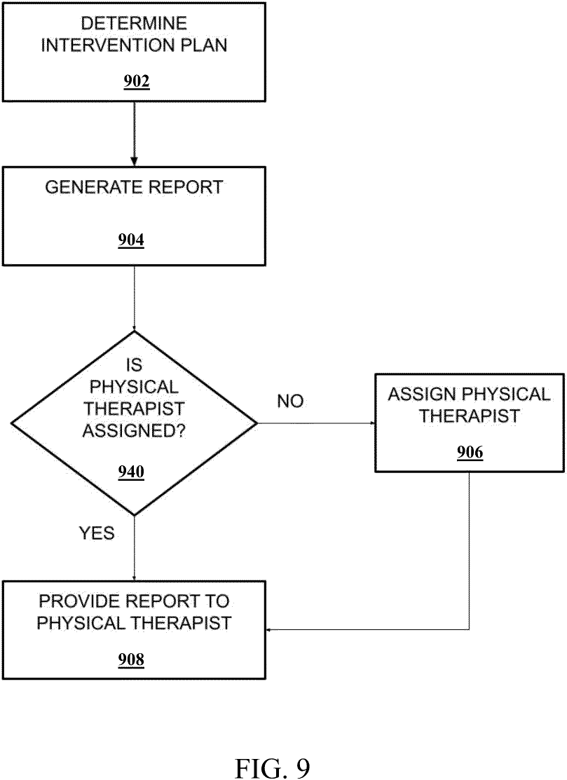

FIG. 9 illustrates an embodiment of a method for self-guided physical therapy and providing a report to a physical therapist. As shown in FIG. 9, the method includes the steps of: determining an intervention plan 902; generating a report 904; and providing the report to a physical therapist. Additionally, if a physical therapist is not assigned to the user, then a physical therapist may be assigned 906.

The method for creating and providing medical documentation 900 is a sub-method that may be present in the method of FIG. 3. In some embodiments, the method to create and provide medical documentation may occur alongside determining the intervention 310 of FIG. 3. In another embodiment, the method 900 may occur at any point during the system and method for determining physical therapy interventions. In some instances, the steps of the method to create and provide medical documentation 900 may be disbursed within FIG. 3.

In one embodiment, the report may be a list of assigned interventions. In another embodiment, the report may include user’s inputs, such as biographical information, safety screening, and objective and subjective exams. Additionally, the report may include the user’s progress and interaction with the system.

As demonstrated in FIG. 9, the report may be provided to a physical therapist 908. In some instances, the report may be provided to any medical professional, insurance company, attorney, and employer, as non-limiting examples.

The system may additionally allow for coordination between the physical therapist and the user. In one embodiment, the physical therapist may be able to contact the user. In another embodiment, the system may allow for coordination between the physical therapist to arrange an appointment. In some instances, the appointments may be telehealth appointments. In other instances, the appointments may be office appointments. It is contemplated that the system and method for determining physical therapy intervention reduce the overall number of coordinated appointments, thus making the program more accessible.

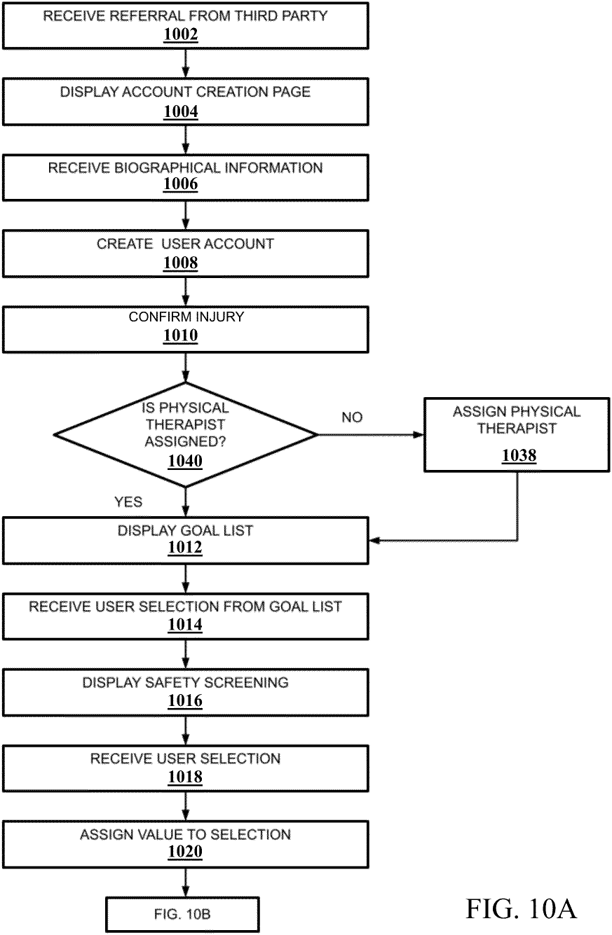

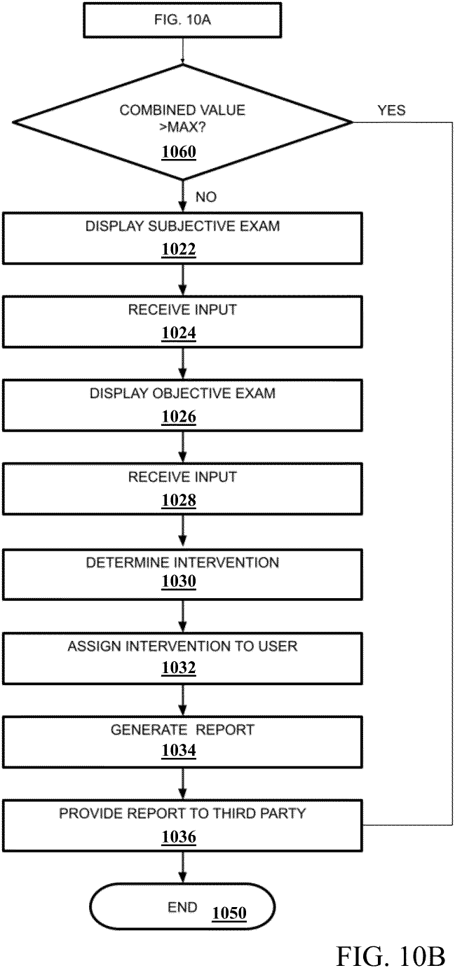

FIG. 10 is a flowchart depicting an embodiment of a method for self-guided physical therapy from a referral, confirming an injury, and providing a report to a third party. As shown in FIG. 10, the method includes the steps of: receiving a referral from a third party 1002; displaying an account creation page 1004; receiving biographical information 1006; creating a user account 1008; confirming an injury 1010; assigning a physical therapist if no physical therapist is assigned 1038 and 1040; displaying a goal list 1012; receiving user selection from the goal list 1014; displaying safety screening questions 1016; receiving the user’s selection 1018; assigning a value to each selection 1020; if the value of the selection is less than a predetermined threshold 1060 then displaying a subjective exam 1022; receiving user’s input 1024; displaying an objective exam 1026; receiving user’s input 1028; determining an intervention 1030; assigning an intervention to the user 1032; generating a report 1034; and providing that report to a third party 1036. In some instances, when the value of the selection is equal to or greater than a predetermined threshold 1060 a report may be provided to the third party.

In some instances, the third party that provides the referral 1002 and the third party that receives the report 1036 may be the same person. In other instances, the third party that provides the referral 1002 and the third party that receives the report 1036 may be different persons. Additionally, the third party 1002 or 1036 may be the same as the physical therapist 1040. In some instances, the third party 1002 and 1036 may refer to the physical therapist 1040.

In one embodiment, confirming an injury 1010 includes displaying questions based on the referral and receiving the user’s selection. More particularly, confirming an injury 1010 may include displaying questions whose answers authenticate or confirm the reason that the patient is using or has been referred to use the system. For instance, an injury may be knee, back, shoulder pain or the like, and/or any other affliction or medical condition which may be improved or addressed by physical methods undertaken in physical therapy. As such, confirming an injury 1010 may involve receiving user input that describes or identifies the reason that they are using the system.

In another embodiment, the displayed goals 1012 may relate to the information provided in the referral. In other instances, the displayed goals 1012 may not relate to the information provided in the referral.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, in some embodiments physical therapy treatment may occur both in office and remote, reducing the burden on both the patient and physical therapist while providing a more conventional treatment approach. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the system and method for self-guided physical therapy with which that terminology is associated. In general, the terms used in the following claims should not be constructed to limit the system and method for self-guided physical therapy to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the system and method for self-guided physical therapy is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the system and method for self-guided physical therapy.

What is claimed is:

1. A system for self-guided physical therapy comprising:
a first computing device operative to:
a. receive biographical information;
b. generate a user account corresponding to the biographical information;
c. display a set of user-specific screening parameters;
d. receive at least one selection of user-specific screening parameters; and
e. assign an intervention to the user account, wherein the intervention correlates to the selection.

2. The system of claim 1, further comprising a second computing device, wherein the first computing device is further operative to receive a referral from the second computing device.

3. The system of claim 1, wherein the first computing device is further operative to authenticate that a user matches the referral by displaying a confirmation question.

4. The system of claim 1, wherein said user-specific screening parameters comprise a set of objective parameters, wherein the objective parameters are selected from a group consisting of range of motion, flexibility, strength, size, and combinations thereof.

5. The system of claim 1, wherein said user-specific screening parameters comprise a set of subjective parameters, wherein the subjective parameters are selected from a group consisting of pain location, actions that give rise to increased symptoms, time elapsed since onset of injury, pain level, and combinations thereof.

6. The system of claim 1, wherein said user-specific screening parameters comprise a set of safety parameters, wherein the safety parameters are selected from a group consisting of injury history, temperature, biological changes, and combinations thereof.

7. The system of claim 1, wherein the first computing device is further operative to assign a value to each of the at least one selections, and for a combined value greater than a predetermined threshold, prevent further access to the system.

8. The system of claim 1, wherein the first computing device is further operative to assign a physical therapist.

9. The system of claim 1, wherein the first computing device is further operative to generate a report comprising the at least one selection and the intervention.

10. The system of claim 9, further comprising a second computing device, wherein the first computing device is further operative to send the report to the second computing device.

11. The system of claim 1, wherein the first computing device is further operative to display the parameters periodically and repeat steps (c) through (e).

12. The system of claim 1, wherein the first computing device is further operative to receive a set of user goals and assign interventions based on the user goals.

13. The system of claim 1, wherein the first computing device comprises a graphical user interface, wherein the graphical user interface is configured to receive and display information.

14. A system for self-guided physical therapy comprising:
a first computing device operative to:
a. receive a referral from a second computing device;
b. receive biographical information;
c. authenticate that the user information matches the referral;
d. generate an account corresponding to the user information;
e. display a set of user-specific screening parameters, wherein the user-specific screening parameters are selected from a group consisting of objective parameters, subjective parameters, safety screening parameters, and combinations thereof;
f. receive a selection of the user-specific screening parameters;
g. responsive to receiving the selection of user-specific screening parameters, assign an intervention to the user account, wherein the intervention correlates to the parameters and the referral;
h. generate a report comprising the selection and the intervention; and
i. transmit the report to a third-party computing device,
wherein the first computing device comprises a graphical user interface configured to receive and display information.

15. The system of claim 14, wherein the first computing device is further operative to correlate interventions to one or more goals and assign one or more interventions responsive to receiving one or more goals from the user.

16. The system of claim 14, wherein said mobile device is further operative to periodically display the parameters and repeat steps (e) through (i).

17. A non-transitory, tangible computer-readable medium having stored thereon computer-executable instructions, which, when executed by a computer processor, enable performance of the method comprising:
by an electronic device, providing a graphical user interface having:
a. an account display configured to receive user input comprising biographical information,
b. a medical condition display configured to receive user input comprising information characterizing at least one medical condition, and
c. a medical intervention display configured to display and enable user to assigned interventions;
receiving at least one medical condition;
adjusting on the graphical user interface the medical intervention portion with respect to the medical condition; and
displaying on the medical intervention portion an intervention.

19. The method of claim 18, further comprising receiving additional medical condition symptoms.
